(19) United States
(12) Patent Application Publication
(10) Pub. No.: US 2011/0002861 A1
(43) Pub. Date: Jan. 6, 2011

(54) COMPOSITION COMPRISING PURE ALOE AND ITS USE AS A COSMETIC BASE

(76) Inventor: Stefano Sala, Trento (IT)

Correspondence Address: MODIANO & ASSOCIATI, Via Meravigli 16, Milano, 20123, IT

(21) Appl. No.: 12/736,054

(22) PCT Filed: Mar. 12, 2009
(86) PCT No.: PCT/EP2009/052946

§ 371 Date: Sep. 3, 2010

(30) Foreign Application Priority Data
Mar. 14, 2008 (IT) PD2008A000084

(51) Int. Cl. A61K 36/886; A61K 8/97; A61P 31/00; A61Q 17/04; A61P 17/00; A61Q 1/02; A61Q 13/00; A61Q 15/00; A61P 17/18; A61P 17/08

(52) U.S. Cl. 424/59; 424/63; 424/126; 424/744

(57) ABSTRACT

A composition to be used as a base for cosmetic products, comprising aloe gel from 50% to 99% by weight and an emulsifier comprising an acryloyl taurate copolymer, a non-ionic surfactant consisting of an ester of sorbitol polyethoxylate, and an oil of natural origin. The use of the composition described here for the preparation of a cosmetic product and a cosmetic product comprising the composition described here are also provided.

The present invention relates to a composition comprising aloe that is useful for preparing cosmetic products for external use.

### BACKGROUND OF THE INVENTION

Aloe is a so-called succulent plant, which belongs to the Asphodelaceae family and is native of some African regions, although it is now common in many Mediterranean countries, in the Middle East, in Mexico and in the southern United States. Like all succulent plants, aloe is characterized by the presence of particular plant tissues, known as aquiferous parenchyma, within which the plant is able to absorb large quantities of water, so as to be able to survive in the dry climates of its regions of origin and in drought periods.

There are many varieties of aloe, but the best-known one is probably Aloe barbadensis Miller or Aloe vera. The notoriety of Aloe vera is due to its use in the medicinal and cosmetic field, which apparently is known since past times. Aloe is in fact said to have, among others, moisturizing, anti-inflammatory, lenitive, skin normalizing, wound healing, immunomodulating and laxative properties. These properties are derived from the substances contained mainly in two aloe derivatives, the exudate and the gel.

The exudate is a yellow resinous liquid, which is present on the outside of aloe leaves, and is particularly rich in anthraquinone compounds, to which the laxative action is mostly due. The main anthraquinones that are present in the aloe plant are aloin, aloemodin and barbaloin. Aloe gel instead represents the gelatinous substance that is naturally contained in aloe leaves. This gelatinous substance is constituted mostly by water, polysaccharides, fibers, proteins, and also contains a small quantity of anthraquinone compounds. The main polysaccharides contained in the gel are glucans, glucomannans, acemannans and so-called mucilages, polysaccharides that contain monomeric units of xylose. It is believed that the properties of aloe mentioned earlier are mostly due to polysaccharides and anthraquinones.

Aloe gel, therefore, is a source of compounds with various beneficial actions, whose use is currently widespread in the preparation of products for cosmetic use of various kinds. There are in fact various types of product that contain aloe gel, including products in the form of gel and products in the form of cream.

These products differ not only in their cosmetic form, but also in the aloe content that can characterize them. In fact, while gels can have high concentrations of aloe, in creams the aloe content is lower than in gels.

Generally, in the preparation of aloe-based compositions, the aloe gel is dispersed in an aqueous phase, and therefore the higher the content of aqueous phase of a cosmetic product, the higher the content of aloe inside it. Creams are constituted by emulsions, i.e., heterogeneous, disperse systems constituted by two mutually immiscible liquid phases, an aqueous one and an oily one. In order to obtain a cream, therefore, it is not possible to use a composition in which the aqueous phase has an excessive volume and accordingly the quantity of aloe is in turn limited.

In particular, in currently commercially available creams the aloe content never exceeds 40-50% by weight, since otherwise, i.e., at higher concentrations, the resulting product would not have a creamy appearance, but rather a consistency that would make it more similar to a gel.

Therefore, currently, cosmetic products in the form of cream are characterized by the need to add water and/or by a large quantity of emulsifying agents. Accordingly, these products either contain limited quantities of aloe, and are therefore scarcely effective in providing the user with the beneficial properties of aloe, or have a limited consistency, are excessively liquid and are therefore difficult to apply. For example, in the case of application to the skin, it is observed that due to the limited consistency these products tend to glide on the skin, being difficult to spread and absorb.

There is, therefore, the need to provide a composition comprising aloe gel in a large quantity that is characterized by a superior consistency with respect to known compositions and that can be included within cosmetic products for external use.

### SUMMARY OF THE INVENTION

The aim of the present invention is to provide a new composition for cosmetic products that contains a large quantity of aloe gel.

Within this aim, an object of the invention is to provide a composition that can enhance the moisturizing and lenitive characteristics of aloe.

Another object of the invention is to provide a composition comprising aloe gel that can be used as a cosmetic base without the need to add water.

Another object of the present invention is to provide a composition for cosmetic products for external use that has a natural base such as aloe gel.

Another object of the present invention is to provide a composition comprising aloe gel by means of which it is possible to obtain various forms of cosmetic product.

Another object of the invention is to provide a composition comprising aloe gel that is useful as a cosmetic base and is highly reliable, relatively easy to provide and at competitive costs.

This aim and these and other objects, which will become better apparent hereinafter, are achieved by a composition comprising:

a) aloe gel in a quantity from 50% to 99% by weight of the composition, and

b) an emulsifier comprising an acryloyl taurate copolymer, a non-ionic surfactant consisting of an ester of sorbitol polyethoxylate, and an oil of natural origin.

The aim and the objects of the invention are also achieved by the use of the composition described here to prepare a cosmetic product for external use.

Furthermore, the aim and the objects of the invention are also achieved by a cosmetic product for external use which comprises the composition described herein.

### DESCRIPTION OF THE PREFERRED EMBODIMENTS

Further objects, characteristics and advantages of the invention will be described in the following detailed description.

According to the invention, the composition comprises a large quantity of aloe gel. Aloe gel is particularly rich in polysaccharides and also contains a small quantity of anthraquinone compounds, to which the beneficial properties of the plant are mostly due. By formulating a composition containing a large concentration of aloe gel, therefore, a higher concentration of the substances that have a curative action is obtained.

In the composition according to the invention, the aloe gel is comprised in a quantity from 50% to 99% by weight of the composition. More preferably, the aloe gel is in a quantity from 70% to 99% by weight of the composition. Even more preferably, the aloe gel is in a quantity from 93% to 97% by weight of the composition.

The second component of the composition according to the present invention is an emulsifying product, by way of which it is possible to obtain a formulation that has a high aloe content. In fact, as previously mentioned, known aloe-based compositions cannot contain a quantity of aloe beyond 40-50% by weight without compromising the consistency of the product.

According to the invention, the composition comprises an emulsifier that comprises an acryloyl taurate copolymer, a non-ionic surfactant consisting of an ester of sorbitol polyethoxylate, and an oil of natural origin, by way of which it is possible to obtain a formulation with a high quantity of aloe without negatively affecting the consistency of the product.

Preferably, the composition comprises the emulsifier in a quantity from 1% to 50% by weight of the composition, more preferably from 1% to 30% by weight, even more preferably from 3% to 7% by weight.

In a preferred embodiment, in the formulation of the emulsifier the acryloyl taurate copolymer is a copolymer of hydroxyethyl acrylate and sodium acryloyldimethyl taurate, the non-ionic surfactant is polysorbate 60 and the oil of natural origin is squalane.

In the present context, the expression “copolymer of hydroxyethyl acrylate and sodium acryloyldimethyl taurate” refers to a polymer prepared by copolymerization of a monomer such as sodium acryloyldimethyl taurate with at least one second monomer that consists of 2-hydroxyethyl acrylate. The term “polysorbate 60” refers to a surfactant of non-ionic type consisting of sorbitol esterified with stearic acid and ethoxylated, also known as polyoxyethylene (20) sorbitan monostearate (sorbitan monostearate ethoxylated with 20 moles of ethylene oxide). The term “squalane” refers to a chemical compound also known as perhydrosqualene or 2,6,10,15,19,23-hexamethyltetracosane, obtained from natural sources, particularly from olives.

In another preferred embodiment, the emulsifier comprises the copolymer of hydroxyethyl acrylate and sodium acryloyldimethyl taurate in a quantity from 20% to 60% by weight of the emulsifier, more preferably from 25% to 45% by weight, polysorbate 60 in a quantity from 2.5% to 15% by weight of the emulsifier, more preferably from 4% to 9% by weight, and squalane in a quantity from 15% to 40% by weight of the emulsifier, more preferably from 20% to 25% by weight.

Further, in another embodiment the composition according to the invention is characterized in that it is not further added with water.

Since the composition according to the invention can be used as a base for formulating cosmetic products, it may or may not contain additional ingredients. In particular, the composition according to the invention can be characterized in that it further comprises at least one cosmetic active ingredient.

In one embodiment of the present invention, the cosmetic active ingredient is selected from the group consisting of vitamins (for example vitamin A or vitamin E), minerals, moisturizing factors, tensor-effect factors, plant factors with a lenitive effect, essential oils, re-epithelizing and structurant factors, and silicones.

The expression “moisturizing factors” refers to substances capable of maintaining and re-establishing the correct degree of moisture of the skin, such as for example hyaluronic acid.

The expression “tensor-effect factors” refers to substances capable of providing a lifting action on the epithelial tissue, such as for example hydroxypropyl beta-cyclodextrins.

The expression “plant factors with lenitive action” refers to emollient substances of natural origin capable of alleviating skin irritations, such as for example extract of calendula, extract of camomile, extract of mallow or fermented papaya.

The expression “essential oils” refers to liquid substances of plant origin characterized by a hydrophobic nature and consisting of non-hydrosoluble volatile aromatic compounds. Essential oils have, among others, stimulating, antiseptic and epithelial tissue healing medicinal properties. For example, the essential oils may be essential oil of lavender or essential oil of tea-tree.

The expression “re-epithelizing and structurant factors” is used to refer to substances capable of stimulating the regrowth capacities of the epithelial tissues, such as for example collagen.

The term “silicones” refers to polysiloxanes, polymeric substances characterized by a mixed nature of organic and inorganic type with a wetting and lubricating action, so as to improve the feel of the composition when applied to the skin (skin feel). For example, the silicones may be dimethicone and cyclomethicone.

Of course, the composition according to the invention may further comprise one or more additives commonly used in the field of cosmetics. For example, such additives can be selected from the group consisting of preservatives, surfactants, fragrances, pigments and mixtures thereof.

For example, the preservatives may be selected from the group consisting of citric acid, sodium benzoate, potassium sorbate, phenoxyethanol, benzoic acid, dehydroacetic acid and mixtures thereof. The surfactants can instead be selected from the group consisting of sodium dodecyl sulfate, sodium laureth sulfate, benzalkonium chloride, cocamidopropyl betaine and cocamide ethanolamine.

Further additives used in the cosmetic field and that the composition according to the invention may further comprise may be selected from the group consisting of a tanning agent, an abrasive agent, a buffering agent, an anticorrosion agent, an antimicrobial agent, an antioxidant, an antiseborrheic agent, an antiperspirant, an absorbent, a UV absorbent, an astringent, a chelating agent, a keratolytic agent, a skin conditioner, a deodorant, a depigmenting agent, an emollient, a film-forming agent, a UV filter, a moisturizing agent, a bonding agent, a refinisher, an opacifier, a pearlescer, a skin protectant, a cooling agent, a refatting agent, a stabilizer, a toner and mixtures thereof.

The expression “antiseborrheic” refers to substances capable of reducing the cutaneous excess of sebaceous secretion, such as for example urea, coriander (coriandrum sativum) or hydroxyphenyl glycinamide.

The term “antiperspirant” makes reference to substances capable of limiting excessive local perspiration of the skin, such as for example potassium alum, sage (salvia officinalis) or aluminum salts.

The term “absorbent” refers to substances capable of applying an absorbent action on compounds that are dissolved or finely dispersed and are soluble in water and/or in oil. For example, the absorbent may be selected from the group consisting of calcium salts, cellulose, aluminosilicate, starch and bentonite.

The term “astringent” makes reference to substances that are capable of reducing tissue secretions, such as for example horsetail (equisetum arvense), witch hazel (hamamelis virginiana) or iron salts.

The term “keratolytic” refers to substances capable of facilitating the elimination of dead cells of the horny layer of the epithelium, such as for example salicylamide, salicylic acid, tamarind (tamarindus indica) or enzymes.

The expression “skin conditioner” refers to substances capable of maintaining the balance of the epithelial tissue and facilitating its permanence in a good condition, such as for example vitamin A, vitamin E, elastin, glycerides, acetylglutamines or acetylglucosamines.

The expression “film forming agent” makes reference to substances capable of leading to the formation of a layer of polymeric material (patina) where applied, such as for example beeswax, acrylamide or acrylate copolymers.

The expression “bonding agent” refers to substances capable of facilitating the cohesion among the components of a cosmetic composition, such as for example agar, sodium alginate, alginic acid, carrageenan or isopropyl palmitate.

The term “refinisher” makes reference to substances capable of performing a delicate exfoliative action on the skin, with the effect of making the skin smoother. For example, the refinisher may be selected from the group consisting of mallow (malva silvestris), propolis and sericin.

The term “opacifier” refers to substances capable of reducing the transparent and translucent appearance of a cosmetic composition by increasing its opacity, such as for example aluminum salts, isostearic acid, myristic acid, palmitic acid or microcrystalline wax.

The term “pearlescer” makes reference to substances capable of giving a pearly appearance to the cosmetic compositions in which they are inserted and are for example calcium salts or salicylic acid.

The expression “skin protectant” refers to substances capable of facilitating the protection of the skin against the damaging action of external agents, such as for example urea, hydrolyzed collagen, titanium oxide or chlorogenic acid.

The expression “refatting agent” makes reference to substances capable of restoring the fatty content of hair and of the upper layers of the skin, such as for example karite butter or borage oil.

The term “stabilizer” refers to substances whose addition to the cosmetic compositions facilitates the emulsifying process and improves the stability and preservation of the composition. For example, the stabilizer may be carboxymethyl cellulose (CMC), carboxymethyl hydroxyethyl cellulose, hydroxyethyl cellulose, guar gum, glucose oxidase or cocoamides.

The invention further consists of a cosmetic product for external use that comprises the composition according to the invention. Such cosmetic product may assume various forms suitable for external use, including the forms of cream, emulsion, gel, milk, suspension, lotion or soap. Preferably, the cosmetic product may be in the form of a cream.

The present invention is now described by means of the following example, which must be considered purely as a non-limiting illustration of the aim and objects of the invention.

A composition according to the invention was formulated by using, according to the proportions shown in Table 1, aloe gel, emulsifier and, as additives, a packet of preservatives comprising citric acid, sodium benzoate, potassium sorbate, phenoxyethanol, benzoic acid and dehydroacetic acid.

TABLE 1

| Ingredient | INCI name* | Quantity (% weight) |
|---|---|---|
| Aloe gel | Aloe barbadensis gel | 96% |
| Emulsifier | hydroxyethyl acrylate/sodium | 3.2% |
| | acryloyldimethyl taurate copolymer; | |
| | squalane; polysorbate 60 | |
| Preservatives | citric acid; sodium benzoate; | 0.8% |
| | potassium sorbate; phenoxylethanol; | |
| | benzoic acid; dehydroacetic acid | |

*INCI = International Nomenclature of Cosmetic Ingredients.

The composition formulated according to Table 1 was analyzed to determine its chemical and physical properties in terms of pH, viscosity and density. The resulting values are indicated in Table 2, (where “n/a” indicates “not applicable”).

TABLE 2

| Property | Unit of measure | Value |
|---|---|---|
| pH | n/a | 1-8 |
| Viscosity | MPa · s | 1000-8000 |
| Density | g/cm³ | 0.990-1.009 |

The composition thus prepared is characterized by a high content of aloe and has an adequate consistency, which makes it suitable for preparing cosmetic products for external use and in particular products in the form of creams.

In practice it has been found that the composition according to the invention fully achieves the intended aim, since by combining the aloe gel in a quantity of more than 50% with an emulsifier that comprises an acryloyl taurate copolymer, a non-ionic surfactant consisting of an ester of sorbitol polyethoxylate and an oil of national origin, a composition characterized by a high content of aloe gel and a high consistency to the touch is obtained.

Further, it has been observed that the composition according to the invention allows to enhance the moisturizing and lenitive characteristics of aloe by way of the possibility to use a quantity of aloe of more than 50% by weight.

It has also been observed that the composition according to the invention is suitable for preparing various forms of cosmetic products for external use.

The composition and the cosmetic product thus conceived are susceptible of numerous modifications and variations, all of which are within the scope of the appended claims; all the details may further be replaced with other technically equivalent elements.

The disclosures in Italian Patent Application No. PD2008A000084 from which this application claims priority are incorporated herein by reference.

1-12. (canceled)

13. A composition comprising:
a) aloe gel in a quantity from 50% to 99% by weight of the composition, and
b) an emulsifier comprising an acryloyl taurate copolymer, a non-ionic surfactant consisting of an ester of sorbitol polyethoxylate, and an oil of natural origin.

14. The composition according to claim 13, wherein the aloe gel is in a quantity from 70% to 99% by weight of the composition.

15. The composition according to claim 13, wherein the emulsifier is comprised in a quantity from 1% to 50% by weight of the composition.

16. The composition according to claim 13, wherein the acryloyl taurate copolymer is a copolymer of hydroxyethyl acrylate and sodium acryloyldimethyl taurate, the non-ionic surfactant is polysorbate 60, and the natural oil is squalane.

17. The composition according to claim 16, wherein the emulsifier comprises the copolymer of hydroxyethyl acrylate and sodium acryloyldimethyl taurate in a quantity from 20% to 60% by weight of the emulsifier, polysorbate 60 in a quantity from 2.5% to 15% by weight of the emulsifier, and squalane in a quantity from 15% to 40% by weight of the emulsifier.

18. The composition according to claim 13, wherein it is not added with water.

19. The composition according to claim 13, characterized in that it further comprises at least one cosmetic active ingredient.

20. The composition according to claim 13, further comprising one or more additives selected from the group consisting of a preservative, a surfactant, a fragrance, a pigment, a tanning agent, an abrasive agent, a buffering agent, an anticorrosion agent, an antimicrobial agent, an antioxidant, an antiseborrheic agent, an antiperspirant, an absorbent, a UV absorbent, an astringent, a chelating agent, a keratolytic agent, a skin conditioner, a deodorant, a depigmenting agent, an emollient, a film-forming agent, a UV filter, a moisturizing agent, a bonding agent, a refinisher, an opacifier, a pearlescer, a skin protectant, a cooling agent, a refatting agent, a stabilizer, a toner and mixtures thereof.

21. Use of the composition according to claim 13 to prepare a cosmetic product for external use.

22. A cosmetic product for external use comprising the composition according to claim 13.

23. The cosmetic product according to claim 22, wherein it is in the form of a cream, a gel, an emulsion, a milk, a suspension, a lotion or a soap.

24. The cosmetic product according to claim 23, wherein it is in the form of a cream.
